SEQUENCE LISTING (36 sequences)

SEQ ID NO: 1 - protein, 904 aa, Herpes simplex virus (type 1 / strain 17)
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 2 - protein, 874 aa, Herpes simplex virus (type 1 / strain 17)
APSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 3 - protein, 904 aa, Artificial Sequence
  gB pre-protein His516Pro
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 4 - protein, 874 aa, Artificial Sequence
  gB mature protein His486Pro
APSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 5 - protein, 904 aa, Artificial Sequence
  gB pre-protein Ser392Cys+Gln532Cys
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAICTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELCNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 6 - protein, 874 aa, Artificial Sequence
  gB mature protein Ser362Cys+Gln502Cys HSV1
APSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAICTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELCNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 7 - protein, 904 aa, Artificial Sequence
  gB pre-protein His516Pro+Ser392Cys+Gln532Cys
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAICTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELCNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 8 - protein, 874 aa, Artificial Sequence
  gB mature protein His486Pro+Ser362Cys+Gln502Cys HSV1
APSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAICTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELCNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 9 - protein, 857 aa, Epstein-Barr virus (strain B95-8)
  gB pre-protein EBV
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 10 - protein, 836 aa, Epstein-Barr virus (strain B95-8)
  gB mature protein EBV
AQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 11 - protein, 857 aa, Epstein-Barr virus (strain B95-8)
  gB pre-protein Gln471Pro EBV
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRPINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 12 - protein, 836 aa, Epstein-Barr virus (strain B95-8)
  gB mature protein Gln450Pro EBV
AQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRPINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 13 - protein, 931 aa, Varicella-zoster virus (strain Oka vaccine)
  gB pre-protein VZV
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 14 - protein, 860 aa, Varicella-zoster virus (strain Oka vaccine)
  gB mature protein VZV
VSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 15 - protein, 931 aa, Varicella-zoster virus (strain Oka vaccine)
  gB pre-protein His527Pro VZV
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEPVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 16 - protein, 860 aa, Varicella-zoster virus (strain Oka vaccine)
  gB mature protein His456Pro VZV
VSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEPVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 17 - protein, 931 aa, Varicella-zoster virus (strain Oka vaccine)
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLCTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLCNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 18 - protein, 860 aa, Varicella-zoster virus (strain Oka vaccine)
  gB mature protein Ser326Cys+Gln472Cys VZV
VSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLCTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLCNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 19 - protein, 931 aa, Varicella-zoster virus (strain Oka vaccine)
  gB pre-protein His527Pro+Ser397Cys+Gln543Cys VZV
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLCTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEPVNEMLARISSSWCQLCNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 20 - protein, 860 aa, Varicella-zoster virus (strain Oka vaccine)
  gB mature protein His456Pro+Ser326Cys+Gln472Cys VZV
VSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLCTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEPVNEMLARISSSWCQLCNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 21 - protein, 907 aa, Cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 22 - protein, 883 aa, Cytomegalovirus
SSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 23 - protein, 907 aa, Cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGPINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 24 - protein, 883 aa, Cytomegalovirus
  gB mature protein Tyr470Pro HCMV
SSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGPINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 25 - protein, 830 aa, Human herpesvirus 6A (strain Uganda-1102)
  gB pre-protein HHV6
MSKMAVLFLAVFLMNSVLMIYCDPDHYIRAGYNHKYPFRICSIAKGTDLMRFDRDISCSPYKSNAKMSEGFFIIYKTNIETYTFPVRTYKKELTFQSSYRDVGVVYFLDRTVMGLAMPVYEANLVNSHAQCYSAVAMKRPDGTVFSAFHEDNNKNNTLNLFPLNFKSITNKRFITTKEPYFARGPLWLYSTSTSLNCIVTEATAKAKYPFSYFALTTGEIVEGSPFFNGSNGKHFAEPLEKLTILENYTMIEDLMNGMNGATTLVRKIAFLEKADTLFSWEIKEENESVCMLKHWTTVTHGLRAETNETYHFISKELTAAFVAPKESLNLTDPKQTCIKNEFEKIINEVYMSDYNDTYSMNGSYQIFKTTGDLILIWQPLVQKSLMFLEQGSEKIRRRRDVGDVKSRHDILYVQLQYLYDTLKDYINDALGNLAESWCLDQKRTITMLHELSKISPSSIVSEVYGRPISAQLHGDVLAISKCIEVNQSSVQLHKSMRVVDAKGVRSETMCYNRPLVTFSFVNSTPEVVPGQLGLDNEILLGDHRTEECEIPSTKIFLSGNHAHVYTDYTHTNSTPIEDIEVLDAFIRLKIDPLENADFKVLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQRHVLSKPIDMMFPYATNPVTTVSSVTGTTVVKTPSVKDVDGGTSVAVSEKEEGMADVSGQVSDDEYSQEDALKMLKAIKSLDESYRRKPSSSESHASKPSLIDRIRYRGYKSVNVEEA

SEQ ID NO: 26 - protein, 808 aa, Human herpesvirus 6A (strain Uganda-1102)
  gB mature protein HHV6
DPDHYIRAGYNHKYPFRICSIAKGTDLMRFDRDISCSPYKSNAKMSEGFFIIYKTNIETYTFPVRTYKKELTFQSSYRDVGVVYFLDRTVMGLAMPVYEANLVNSHAQCYSAVAMKRPDGTVFSAFHEDNNKNNTLNLFPLNFKSITNKRFITTKEPYFARGPLWLYSTSTSLNCIVTEATAKAKYPFSYFALTTGEIVEGSPFFNGSNGKHFAEPLEKLTILENYTMIEDLMNGMNGATTLVRKIAFLEKADTLFSWEIKEENESVCMLKHWTTVTHGLRAETNETYHFISKELTAAFVAPKESLNLTDPKQTCIKNEFEKIINEVYMSDYNDTYSMNGSYQIFKTTGDLILIWQPLVQKSLMFLEQGSEKIRRRRDVGDVKSRHDILYVQLQYLYDTLKDYINDALGNLAESWCLDQKRTITMLHELSKISPSSIVSEVYGRPISAQLHGDVLAISKCIEVNQSSVQLHKSMRVVDAKGVRSETMCYNRPLVTFSFVNSTPEVVPGQLGLDNEILLGDHRTEECEIPSTKIFLSGNHAHVYTDYTHTNSTPIEDIEVLDAFIRLKIDPLENADFKVLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQRHVLSKPIDMMFPYATNPVTTVSSVTGTTVVKTPSVKDVDGGTSVAVSEKEEGMADVSGQVSDDEYSQEDALKMLKAIKSLDESYRRKPSSSESHASKPSLIDRIRYRGYKSVNVEEA

SEQ ID NO: 27 - protein, 830 aa, Human herpesvirus 6A (strain Uganda-1102)
  gB pre-protein Tyr425Pro HHV6
MSKMAVLFLAVFLMNSVLMIYCDPDHYIRAGYNHKYPFRICSIAKGTDLMRFDRDISCSPYKSNAKMSEGFFIIYKTNIETYTFPVRTYKKELTFQSSYRDVGVVYFLDRTVMGLAMPVYEANLVNSHAQCYSAVAMKRPDGTVFSAFHEDNNKNNTLNLFPLNFKSITNKRFITTKEPYFARGPLWLYSTSTSLNCIVTEATAKAKYPFSYFALTTGEIVEGSPFFNGSNGKHFAEPLEKLTILENYTMIEDLMNGMNGATTLVRKIAFLEKADTLFSWEIKEENESVCMLKHWTTVTHGLRAETNETYHFISKELTAAFVAPKESLNLTDPKQTCIKNEFEKIINEVYMSDYNDTYSMNGSYQIFKTTGDLILIWQPLVQKSLMFLEQGSEKIRRRRDVGDVKSRHDILYVQLQYLYDTLKDPINDALGNLAESWCLDQKRTITMLHELSKISPSSIVSEVYGRPISAQLHGDVLAISKCIEVNQSSVQLHKSMRVVDAKGVRSETMCYNRPLVTFSFVNSTPEVVPGQLGLDNEILLGDHRTEECEIPSTKIFLSGNHAHVYTDYTHTNSTPIEDIEVLDAFIRLKIDPLENADFKVLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQRHVLSKPIDMMFPYATNPVTTVSSVTGTTVVKTPSVKDVDGGTSVAVSEKEEGMADVSGQVSDDEYSQEDALKMLKAIKSLDESYRRKPSSSESHASKPSLIDRIRYRGYKSVNVEEA

SEQ ID NO: 28 - protein, 808 aa, Human herpesvirus 6A (strain Uganda-1102)
  gB mature protein Tyr403Pro HHV6
DPDHYIRAGYNHKYPFRICSIAKGTDLMRFDRDISCSPYKSNAKMSEGFFIIYKTNIETYTFPVRTYKKELTFQSSYRDVGVVYFLDRTVMGLAMPVYEANLVNSHAQCYSAVAMKRPDGTVFSAFHEDNNKNNTLNLFPLNFKSITNKRFITTKEPYFARGPLWLYSTSTSLNCIVTEATAKAKYPFSYFALTTGEIVEGSPFFNGSNGKHFAEPLEKLTILENYTMIEDLMNGMNGATTLVRKIAFLEKADTLFSWEIKEENESVCMLKHWTTVTHGLRAETNETYHFISKELTAAFVAPKESLNLTDPKQTCIKNEFEKIINEVYMSDYNDTYSMNGSYQIFKTTGDLILIWQPLVQKSLMFLEQGSEKIRRRRDVGDVKSRHDILYVQLQYLYDTLKDPINDALGNLAESWCLDQKRTITMLHELSKISPSSIVSEVYGRPISAQLHGDVLAISKCIEVNQSSVQLHKSMRVVDAKGVRSETMCYNRPLVTFSFVNSTPEVVPGQLGLDNEILLGDHRTEECEIPSTKIFLSGNHAHVYTDYTHTNSTPIEDIEVLDAFIRLKIDPLENADFKVLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQRHVLSKPIDMMFPYATNPVTTVSSVTGTTVVKTPSVKDVDGGTSVAVSEKEEGMADVSGQVSDDEYSQEDALKMLKAIKSLDESYRRKPSSSESHASKPSLIDRIRYRGYKSVNVEEA

SEQ ID NO: 29 - protein, 822 aa, Human herpesvirus 7 (strain JI)
  gB pre-protein HHV7
MKILFLSVFITFSLQLSLQTEADFVMTGHNQHLPFRICSIATGTDLVRFDREVSCASYGSNIKTTEGILIIYKTKIEAHTFSVRTFKKELTFQTTYRDVGTVYFLDRTVTTLPMPIEEVHMVNTEARCLSSISVKRSEEEEYVAYHKDEYVNKTLDLIPLNFKSDTVRRYITTKEPFLRNGPLWFYSTSTSINCIVTDCIAKTKYPFDFFALSTGETVEGSPFYNGINSKTFNEPTEKILFRNNYTMLKTFDDGSKGNFVTLTKMAFLEKGNTIFSWEVQNEESSICLLKHWMTIPHALRAENANSFHFIAQELTASFVTGKSNYTLSDSKYNCINSNYTSILDEIYQTQYNNSHDKNGSYEIFKTEGDLILIWQPLIQRKLTVLENFSNASRKRRKRELETNKDIVYVQLQYLYDTLKDYINTALGKLAEAWCLNQKRTITVLHELSKISPSGIISAVYGKPMSAKLIGDVLAVSKCIEVNQTSVQLHKSMRLTKDSSYDALRCYSRPLLTYSFANSSKETYLGQLGLDNEILLGNHRTEECEQSNTKIFLSGKFAHIFKDYTYVNSSLITEIEALDAFVDLNIDPLENADFTLLELYTKDELSKANVFDLETILREYNSYKSALHHIETKIATVTPTYIGGIDTFFKGLGALGLGLGAVLGVTAGALGDVVNGVFSFLKNPFGGALTILLTLGVIGLVIFLFLRHKRLAQTPIDILFPYTSKSTNSVLQATQSVQAQVKEPLDSSPPYLKTNKDTEPQGDDITHTNEYSQVEALKMLKAIKLLDESYKKAEIAEAKKSQRPSLLERIQYRGYQKLSTEEL

SEQ ID NO: 30 - protein, 800 aa, Human herpesvirus 7 (strain JI)
  gB mature protein HHV7
DFVMTGHNQHLPFRICSIATGTDLVRFDREVSCASYGSNIKTTEGILIIYKTKIEAHTFSVRTFKKELTFQTTYRDVGTVYFLDRTVTTLPMPIEEVHMVNTEARCLSSISVKRSEEEEYVAYHKDEYVNKTLDLIPLNFKSDTVRRYITTKEPFLRNGPLWFYSTSTSINCIVTDCIAKTKYPFDFFALSTGETVEGSPFYNGINSKTFNEPTEKILFRNNYTMLKTFDDGSKGNFVTLTKMAFLEKGNTIFSWEVQNEESSICLLKHWMTIPHALRAENANSFHFIAQELTASFVTGKSNYTLSDSKYNCINSNYTSILDEIYQTQYNNSHDKNGSYEIFKTEGDLILIWQPLIQRKLTVLENFSNASRKRRKRELETNKDIVYVQLQYLYDTLKDYINTALGKLAEAWCLNQKRTITVLHELSKISPSGIISAVYGKPMSAKLIGDVLAVSKCIEVNQTSVQLHKSMRLTKDSSYDALRCYSRPLLTYSFANSSKETYLGQLGLDNEILLGNHRTEECEQSNTKIFLSGKFAHIFKDYTYVNSSLITEIEALDAFVDLNIDPLENADFTLLELYTKDELSKANVFDLETILREYNSYKSALHHIETKIATVTPTYIGGIDTFFKGLGALGLGLGAVLGVTAGALGDVVNGVFSFLKNPFGGALTILLTLGVIGLVIFLFLRHKRLAQTPIDILFPYTSKSTNSVLQATQSVQAQVKEPLDSSPPYLKTNKDTEPQGDDITHTNEYSQVEALKMLKAIKLLDESYKKAEIAEAKKSQRPSLLERIQYRGYQKLSTEEL

SEQ ID NO: 31 - protein, 822 aa, Human herpesvirus 7 (strain JI)
  gB pre-protein Tyr421Pro HHV7
MKILFLSVFITFSLQLSLQTEADFVMTGHNQHLPFRICSIATGTDLVRFDREVSCASYGSNIKTTEGILIIYKTKIEAHTFSVRTFKKELTFQTTYRDVGTVYFLDRTVTTLPMPIEEVHMVNTEARCLSSISVKRSEEEEYVAYHKDEYVNKTLDLIPLNFKSDTVRRYITTKEPFLRNGPLWFYSTSTSINCIVTDCIAKTKYPFDFFALSTGETVEGSPFYNGINSKTFNEPTEKILFRNNYTMLKTFDDGSKGNFVTLTKMAFLEKGNTIFSWEVQNEESSICLLKHWMTIPHALRAENANSFHFIAQELTASFVTGKSNYTLSDSKYNCINSNYTSILDEIYQTQYNNSHDKNGSYEIFKTEGDLILIWQPLIQRKLTVLENFSNASRKRRKRELETNKDIVYVQLQYLYDTLKDPINTALGKLAEAWCLNQKRTITVLHELSKISPSGIISAVYGKPMSAKLIGDVLAVSKCIEVNQTSVQLHKSMRLTKDSSYDALRCYSRPLLTYSFANSSKETYLGQLGLDNEILLGNHRTEECEQSNTKIFLSGKFAHIFKDYTYVNSSLITEIEALDAFVDLNIDPLENADFTLLELYTKDELSKANVFDLETILREYNSYKSALHHIETKIATVTPTYIGGIDTFFKGLGALGLGLGAVLGVTAGALGDVVNGVFSFLKNPFGGALTILLTLGVIGLVIFLFLRHKRLAQTPIDILFPYTSKSTNSVLQATQSVQAQVKEPLDSSPPYLKTNKDTEPQGDDITHTNEYSQVEALKMLKAIKLLDESYKKAEIAEAKKSQRPSLLERIQYRGYQKLSTEEL

SEQ ID NO: 32 - protein, 800 aa, Human herpesvirus 7 (strain JI)
  gB mature protein Tyr399Pro HHV7
DFVMTGHNQHLPFRICSIATGTDLVRFDREVSCASYGSNIKTTEGILIIYKTKIEAHTFSVRTFKKELTFQTTYRDVGTVYFLDRTVTTLPMPIEEVHMVNTEARCLSSISVKRSEEEEYVAYHKDEYVNKTLDLIPLNFKSDTVRRYITTKEPFLRNGPLWFYSTSTSINCIVTDCIAKTKYPFDFFALSTGETVEGSPFYNGINSKTFNEPTEKILFRNNYTMLKTFDDGSKGNFVTLTKMAFLEKGNTIFSWEVQNEESSICLLKHWMTIPHALRAENANSFHFIAQELTASFVTGKSNYTLSDSKYNCINSNYTSILDEIYQTQYNNSHDKNGSYEIFKTEGDLILIWQPLIQRKLTVLENFSNASRKRRKRELETNKDIVYVQLQYLYDTLKDPINTALGKLAEAWCLNQKRTITVLHELSKISPSGIISAVYGKPMSAKLIGDVLAVSKCIEVNQTSVQLHKSMRLTKDSSYDALRCYSRPLLTYSFANSSKETYLGQLGLDNEILLGNHRTEECEQSNTKIFLSGKFAHIFKDYTYVNSSLITEIEALDAFVDLNIDPLENADFTLLELYTKDELSKANVFDLETILREYNSYKSALHHIETKIATVTPTYIGGIDTFFKGLGALGLGLGAVLGVTAGALGDVVNGVFSFLKNPFGGALTILLTLGVIGLVIFLFLRHKRLAQTPIDILFPYTSKSTNSVLQATQSVQAQVKEPLDSSPPYLKTNKDTEPQGDDITHTNEYSQVEALKMLKAIKLLDESYKKAEIAEAKKSQRPSLLERIQYRGYQKLSTEEL

SEQ ID NO: 33 - protein, 845 aa, Human herpesvirus 8 type P (isolate GK18)
  gB pre-protein HHV8
MTPRSRLATLGTVILLVCFCAGAAHSRGDTFQTSSSPTPPGSSSKAPTKPGEEASGPKSVDFYQFRVCSASITGELFRFNLEQTCPDTKDKYHQEGILLVYKKNIVPHIFKVRRYRKIATSVTVYRGLTESAITNKYELPRPVPLYEISHMDSTYQCFSSMKVNVNGVENTFTDRDDVNTTVFLQPVEGLTDNIQRYFSQPVIYAEPGWFPGIYRVRTTVNCEIVDMIARSAEPYNYFVTSLGDTVEVSPFCYNESSCSTTPSNKNGLSVQVVLNHTVVTYSDRGTSPTPQNRIFVETGAYTLSWASESKTTAVCPLALWKTFPRSIQTTHEDSFHFVANEITATFTAPLTPVANFTDTYSCLTSDINTTLNASKAKLASTHVPNGTVQYFHTTGGLYLVWQPMSAINLTHAQGDSGNPTSSPPPSASPMTTSASRRKRRSASTAAAGGGGSTDNLSYTQLQFAYDKLRDGINQVLEELSRAWCREQVRDNLMWYELSKINPTSVMTAIYGRPVSAKFVGDAISVTECINVDQSSVNIHKSLRTNSKDVCYARPLVTFKFLNSSNLFTGQLGARNEIILTNNQVETCKDTCEHYFITRNETLVYKDYAYLRTINTTDISTLNTFIALNLSFIQNIDFKAIELYSSAEKRLASSVFDLETMFREYNYYTHRLAGLREDLDNTIDMNKERFVRDLSEIVADLGGIGKTVVNVASSVVTLCGSLVTGFINFIKHPLGGMLMIIIVIAIILIIFMLSRRTNTIAQAPVKMIYPDVDRRAPPSGGAPTREEIKNILLGMHQLQQEERQKADDLKKSTPSVFQRTANGLRQRLRGYKPLTQSLDISPETGE

SEQ ID NO: 34 - protein, 819 aa, Human herpesvirus 8 type P (isolate GK18)
  gB mature protein HHV8
RGDTFQTSSSPTPPGSSSKAPTKPGEEASGPKSVDFYQFRVCSASITGELFRFNLEQTCPDTKDKYHQEGILLVYKKNIVPHIFKVRRYRKIATSVTVYRGLTESAITNKYELPRPVPLYEISHMDSTYQCFSSMKVNVNGVENTFTDRDDVNTTVFLQPVEGLTDNIQRYFSQPVIYAEPGWFPGIYRVRTTVNCEIVDMIARSAEPYNYFVTSLGDTVEVSPFCYNESSCSTTPSNKNGLSVQVVLNHTVVTYSDRGTSPTPQNRIFVETGAYTLSWASESKTTAVCPLALWKTFPRSIQTTHEDSFHFVANEITATFTAPLTPVANFTDTYSCLTSDINTTLNASKAKLASTHVPNGTVQYFHTTGGLYLVWQPMSAINLTHAQGDSGNPTSSPPPSASPMTTSASRRKRRSASTAAAGGGGSTDNLSYTQLQFAYDKLRDGINQVLEELSRAWCREQVRDNLMWYELSKINPTSVMTAIYGRPVSAKFVGDAISVTECINVDQSSVNIHKSLRTNSKDVCYARPLVTFKFLNSSNLFTGQLGARNEIILTNNQVETCKDTCEHYFITRNETLVYKDYAYLRTINTTDISTLNTFIALNLSFIQNIDFKAIELYSSAEKRLASSVFDLETMFREYNYYTHRLAGLREDLDNTIDMNKERFVRDLSEIVADLGGIGKTVVNVASSVVTLCGSLVTGFINFIKHPLGGMLMIIIVIAIILIIFMLSRRTNTIAQAPVKMIYPDVDRRAPPSGGAPTREEIKNILLGMHQLQQEERQKADDLKKSTPSVFQRTANGLRQRLRGYKPLTQSLDISPETGE

SEQ ID NO: 35 - protein, 845 aa, Human herpesvirus 8 type P (isolate GK18)
  gB pre-protein Gly471Pro HHV8
MTPRSRLATLGTVILLVCFCAGAAHSRGDTFQTSSSPTPPGSSSKAPTKPGEEASGPKSVDFYQFRVCSASITGELFRFNLEQTCPDTKDKYHQEGILLVYKKNIVPHIFKVRRYRKIATSVTVYRGLTESAITNKYELPRPVPLYEISHMDSTYQCFSSMKVNVNGVENTFTDRDDVNTTVFLQPVEGLTDNIQRYFSQPVIYAEPGWFPGIYRVRTTVNCEIVDMIARSAEPYNYFVTSLGDTVEVSPFCYNESSCSTTPSNKNGLSVQVVLNHTVVTYSDRGTSPTPQNRIFVETGAYTLSWASESKTTAVCPLALWKTFPRSIQTTHEDSFHFVANEITATFTAPLTPVANFTDTYSCLTSDINTTLNASKAKLASTHVPNGTVQYFHTTGGLYLVWQPMSAINLTHAQGDSGNPTSSPPPSASPMTTSASRRKRRSASTAAAGGGGSTDNLSYTQLQFAYDKLRDPINQVLEELSRAWCREQVRDNLMWYELSKINPTSVMTAIYGRPVSAKFVGDAISVTECINVDQSSVNIHKSLRTNSKDVCYARPLVTFKFLNSSNLFTGQLGARNEIILTNNQVETCKDTCEHYFITRNETLVYKDYAYLRTINTTDISTLNTFIALNLSFIQNIDFKAIELYSSAEKRLASSVFDLETMFREYNYYTHRLAGLREDLDNTIDMNKERFVRDLSEIVADLGGIGKTVVNVASSVVTLCGSLVTGFINFIKHPLGGMLMIIIVIAIILIIFMLSRRTNTIAQAPVKMIYPDVDRRAPPSGGAPTREEIKNILLGMHQLQQEERQKADDLKKSTPSVFQRTANGLRQRLRGYKPLTQSLDISPETGE

SEQ ID NO: 36 - protein, 819 aa, Human herpesvirus 8 type P (isolate GK18)
  gB mature protein Gly445Pro HHV8
RGDTFQTSSSPTPPGSSSKAPTKPGEEASGPKSVDFYQFRVCSASITGELFRFNLEQTCPDTKDKYHQEGILLVYKKNIVPHIFKVRRYRKIATSVTVYRGLTESAITNKYELPRPVPLYEISHMDSTYQCFSSMKVNVNGVENTFTDRDDVNTTVFLQPVEGLTDNIQRYFSQPVIYAEPGWFPGIYRVRTTVNCEIVDMIARSAEPYNYFVTSLGDTVEVSPFCYNESSCSTTPSNKNGLSVQVVLNHTVVTYSDRGTSPTPQNRIFVETGAYTLSWASESKTTAVCPLALWKTFPRSIQTTHEDSFHFVANEITATFTAPLTPVANFTDTYSCLTSDINTTLNASKAKLASTHVPNGTVQYFHTTGGLYLVWQPMSAINLTHAQGDSGNPTSSPPPSASPMTTSASRRKRRSASTAAAGGGGSTDNLSYTQLQFAYDKLRDPINQVLEELSRAWCREQVRDNLMWYELSKINPTSVMTAIYGRPVSAKFVGDAISVTECINVDQSSVNIHKSLRTNSKDVCYARPLVTFKFLNSSNLFTGQLGARNEIILTNNQVETCKDTCEHYFITRNETLVYKDYAYLRTINTTDISTLNTFIALNLSFIQNIDFKAIELYSSAEKRLASSVFDLETMFREYNYYTHRLAGLREDLDNTIDMNKERFVRDLSEIVADLGGIGKTVVNVASSVVTLCGSLVTGFINFIKHPLGGMLMIIIVIAIILIIFMLSRRTNTIAQAPVKMIYPDVDRRAPPSGGAPTREEIKNILLGMHQLQQEERQKADDLKKSTPSVFQRTANGLRQRLRGYKPLTQSLDISPETGE

(19) United States
(12) Patent Application Publication
(10) Pub. No.: US 2023/0096087 A1
(43) Pub. Date: Mar. 30, 2023

(54) PREFUSION-STABILIZED HERPESVIRUS GLYCOPROTEIN-B

(71) Applicant: Heinrich-Pette-Institut Leibniz-Institut für Experimentelle Virologie, Hamburg (DE)

(72) Inventors: Kay Grünewald, Hamburg (DE); Benjamin Vollmer, Hamburg (DE)

(21) Appl. No.: 17/484,583

(22) Filed: Sep. 24, 2021

(51) Int. Cl. A61K 39/245; C12N 7/00

(52) U.S. Cl. CPC A61K 39/245; C12N 7/00; C12N 2710/16043; C12N 2710/16031; C12N 2710/16022

(57) ABSTRACT

The present invention generally relates to the field of herpesviruses and herpesvirus proteins that can be used for the production of antibodies or therapeutic agents, such as vaccines. More specifically, the present invention relates to a modified herpesvirus glycoprotein B that comprises one or more mutations at defined positions in the primary structure of the protein which retain a stable prefusion conformation which is highly advantageous for the production of antibodies or therapeutic agents, such as vaccines. The invention further relates to nucleic acid molecules which encode such a modified herpesvirus glycoprotein B. The invention further provides methods for producing antibodies or therapeutic agents, such as vaccines, which make use of the modified herpesvirus glycoprotein B or a nucleic acid molecule encoding it. Kits comprising the modified herpesvirus glycoprotein B are provided as well. Finally, the invention also relates to the use of the modified herpesvirus glycoprotein B or a nucleic acid molecule encoding same for drug screening.

### SEQUENCE LISTING

The following application contains a Sequence Listing in computer readable format (CRF), which is submitted electronically via EFS-Web as an ASCII text file entitled “SequenceListing.txt,” created on Sep. 24, 2021, as 261,418 bytes. The content of the Sequence Listing CRF is hereby incorporated by reference in its entirety.

### FIELD OF THE INVENTION

The present invention generally relates to the field of herpesviruses and herpesvirus proteins that can be used for the production of antibodies or therapeutic agents, such as vaccines. More specifically, the present invention relates to a modified herpesvirus glycoprotein B that comprises one or more mutations at defined positions in the primary structure of the protein which retain a stable prefusion conformation which is highly advantageous for the production of antibodies or therapeutic agents, such as vaccines. The invention further relates to nucleic acid molecules which encode such a modified herpesvirus glycoprotein B. The invention further provides methods for producing antibodies or therapeutic agents, such as vaccines, which make use of the modified herpesvirus glycoprotein B or a nucleic acid molecule encoding it. Kits comprising the modified herpesvirus glycoprotein B are provided as well. Finally, the invention also relates to the use of the modified herpesvirus glycoprotein B or a nucleic acid molecule encoding same for drug screening.

### BACKGROUND OF THE INVENTION

Herpesviruses present a significant burden to the general public health. There are at least nine different Herpesviruses that infect humans, namely Herpes Simplex Virus 1 and 2 (HSV-1, HSV-2), Varicella-Zoster Virus (VZV), Epstein—Barr virus (EBV), Human Cytomegalovirus (HCMV), Human Herpesvirus 6A and 6B (HHV-6A, HHV-6B), Human Herpesvirus 7 (HHV-7), and Kaposi's Sarcoma-associated Herpesvirus (KSHV).

One of the hallmark characteristics of Herpesviruses is latency, meaning the virus will stay in the body in a latent state for life, concealed from the host immune system. Although latency is symptom free, the virus can reactivate at any time, by often ill-characterised stimuli. VZV causes varicella and can cause shingles via reactivation and despite introduction of a vaccine in 1995 there were still 6400 associated deaths reported globally in 2015 (GBD Mortality and Causes of Death, 2015). HCMV infection is the most common congenital and perinatal viral infection worldwide and the major cause of permanent hearing loss and neurological impairment in new-borns (Cannon & Schmid, 2010; Damato & Winnen, 2002). Strikingly, this affects up to 3% of all new-borns. In addition to higher transmission rates of HIV in HSV-2 infected people, all Herpesviruses can cause significant morbidity and mortality in immunocompromised (AIDS, cancer or autoimmune disease) patients. Due to the ageing population and increased application of immune-suppressing therapies, the number of individuals prone to these infections will rise. Other complications include Burkitt and Hodgkin's lymphoma caused by EBV. Kaposi's sarcoma and primary effusion lymphoma caused by KSHV, means that two of seven known oncoviruses are Herpesviruses. Due to their high prevalence, Herpesviruses pose a constant threat for patients receiving organ transplants. In 2016 an estimated 3.6 billion people (aged 0-49 years) were infected with HSV-1 (prevalence of 63.6% worldwide), while an estimated 17% (aged 15-49 years) had genital HSV-1 or -2 infections, disproportionally affecting women (James et al. 2020). The seroprevalence for HCMV is similarly high, ranging regionally from 45-100% (Zuhair et al. 2019).

Although these viruses are a severe burden to the global public health, there are currently only two vaccines against Herpesviruses available on the market, both of which are directed against VZV. For all other herpesviruses, no effective vaccines are available due to the fact that the development of an appropriate immune response has turned out to be complicated. The membrane surface of all herpesviruses species is studded by more than 10 different kinds of glycoproteins which are mainly necessary for host cell tropism, attachment and infection. As many of these proteins are redundant, formation of neutralising antibodies is usually unsuccessful or only occurs after infection when the virus already resides in a latent state in the host cells. The only essential and structurally highly conserved glycoprotein on the membrane of herpesviruses is glycoprotein B (gB). The herpesviral gB is a structurally conserved, class III membrane fusion protein, composed of α- and β-secondary structure elements, that combines structural features characteristic of class I and II fusion proteins (Backovic & Jardetzky, 2011). It is essential for infection, since it catalyses the fusion process between the viral and the host cell membrane. Only the fusion of these two membranes allows the release of the viral capsid which contains the viral genome into the host cell.

Membrane fusion is achieved via substantial structural rearrangements of gB from a metastable prefusion conformation to a stable postfusion conformation. Prior to infection, gB is present in the metastable prefusion conformation on the surface of the virus membrane. In response to an external signal, a substantial rearrangement occurs in the protein resulting in exposure of hydrophobic regions called fusion loops which are inserted into the target membrane. Subsequently, the protein undergoes additional conformational changes which lead to the energetically favoured postfusion conformation, providing energy to pull the membranes together leading to membrane fusion and subsequently to the release of the viral content.

Although the first gB postfusion conformation structure was determined for Herpes Simplex Virus 1 gB more than 10 years ago (Heldwein et al., 2006), only recently first descriptions of the prefusion form were reported (Zeev-Ben-Mordehai et al., 2016; Fontana et al., 2017). Attempts to determine high-resolution structures by crystallization or single-particle analysis electron cryomicroscopy failed because of its metastability, causing it to readily adopt the postfusion conformation when using purification methods involving membrane anchor or lipid removal (Patrone et al., 2014). This is true for native gB proteins purified directly from viruses (Oliver et al., 2020) as well as recombinantly expressed herpesvirus gB. The prefusion form presents an attractive target for drug and vaccine development, as demonstrated for the class I fusion protein F of respiratory syncytial virus (RSV), for which the most potent neutralizing antibodies target this conformation only.

The stabilization of gB in the prefusion conformation would prove a major step towards the development of vaccines and other antiviral drugs against herpesviruses which are urgently needed. The present invention solves this problem and provides additional advantages as well.

### DESCRIPTION OF THE INVENTION

It has now been surprisingly found that certain specific mutations in the gB of herpesvirus effectively stabilize the prefusion conformation of the protein. Accordingly, the invention provides for the first time a method to produce recombinant gB proteins in their prefusion conformation which are highly useful for the development of antiviral therapeutics, such as vaccines and compounds that effectively block virus binding.

Therefore, in a first aspect the present invention refers to a modified herpesvirus gB, wherein the protein comprises a mutation in its amino acid sequence which stabilizes the prefusion confirmation of the protein by preventing the irreversible transition from the metastable prefusion to the stable postfusion conformation. The mutation can be a substitution of one or more amino acids in the naturally occurring amino acid sequence of the respective herpesvirus gB. It is particularly preferred, that the herpesvirus gB is a Herpes Simplex gB, such as the gB of HSV-1 or HSV-2. In one embodiment, the modified herpesvirus gB is derived from HSV-1. In another embodiment, the modified herpesvirus gB is derived from HSV-2. In another embodiment, the modified herpesvirus gB is derived from VZV. In another embodiment, the modified herpesvirus gB is derived from The gB of HSV-1 in its immature form (i.e. the pre-protein with the signal peptide ranging from aa 1-30) is depicted in SEQ ID NO:1. The gB of HSV-1 in its mature form (i.e. the processed protein without the signal peptide) is depicted in SEQ ID NO:2.

It has been found in the course of the invention that a particularly suitable modified gB which is locked in the prefusion confirmation can be obtained by mutation of the His residue in the position corresponding to position 516 of the unprocessed HSV-1 envelope gB set forth in SEQ ID NO:1. This His residue is located in position 486 of the mature, processed HSV-1 envelope gB set forth in SEQ ID NO:2. It appears that this residue is located in a potential “hinge” region which plays a decisive role during membrane fusion.

The mutation of the amino acid in position 516 of SEQ ID NO:1 can be a substitution of the amino acid. Preferably, the His residue in the position corresponding to position 516 of SEQ ID NO:1 has been replaced by another amino acid residue. The amino acid which replaces the His residue is preferably selected from the group of non-polar amino acids comprising glycine, valine, alanine, isoleucine, leucine, methionine, proline, phenylalanine, and tryptophan. A substitution of His residue by proline is particularly preferred.

A modified immature gB of HSV-1 carrying a His516Pro substitution is depicted in SEQ ID NO:3. The corresponding mature form of the protein, including the same substitution, is depicted in SEQ ID NO:4. In one embodiment, the modified herpesvirus envelope gB of the invention comprises
(a) the amino acid sequence of SEQ ID NO:3 or SEQ ID NO:4;
(b) an amino acid sequence which has at least 90% identity to the amino acid sequence of SEQ ID NO:3 or SEQ ID NO:4;
(c) an immunologically active fragment of any of (a) or (b).

In a particularly preferred embodiment, the modified herpesvirus envelope gB comprises or consists of the amino acid sequence of SEQ ID NO:3. In yet another particularly preferred embodiment, the modified herpesvirus envelope gB comprises or consists of the amino acid sequence of SEQ ID NO:4.

Since the residue corresponding to the amino acid in position 516 of SEQ ID NO:1 is highly conserved within the group of herpesviruses, it can be readily identified also in other members of this group. For example, the residue corresponding to the amino acid in position 516 of the gB amino acid sequence of HSV-1 can be found in amino acid position 471 of the unprocessed gB of EBV and amino acid position 450 of the processed gB of EBV. The amino acid sequence of the unprocessed gB of EBV is depicted in SEQ ID NO:9. The amino acid sequence of the mature gB of EBV is depicted in SEQ ID NO:10. A modified immature gB of EBV carrying a Gln471Pro substitution is depicted in SEQ ID NO:11. The corresponding mature form of the protein, including the same substitution in position 450, is depicted in SEQ ID NO:12.

Further, the residue corresponding to the amino acid in position 516 of the gB amino acid sequence of HSV-1 can be found in amino acid position 527 of the unprocessed gB of VZV and amino acid position 456 of the processed gB of VZV. The amino acid sequence of the unprocessed gB of VZV is depicted in SEQ ID NO:13. The amino acid sequence of the mature gB of VZV is depicted in SEQ ID NO:14. A modified immature gB of VZV carrying a His527Pro substitution is depicted in SEQ ID NO:15. The corresponding mature form of the protein, including the same substitution in position 456, is depicted in SEQ ID NO:16.

Further, the residue corresponding to the amino acid in position 516 of the gB amino acid sequence of HSV-1 can be found in amino acid position 494 of the unprocessed gB of HCMV and amino acid position 470 of the processed gB of HCMV. The amino acid sequence of the unprocessed gB of HCMV is depicted in SEQ ID NO:21. The amino acid sequence of the mature gB of HCMV is depicted in SEQ ID NO:22. A modified immature gB of HCMV carrying a Tyr494Pro substitution is depicted in SEQ ID NO:23. The corresponding mature form of the protein, including the same substitution in position 470, is depicted in SEQ ID NO:24.

Further, the residue corresponding to the amino acid in position 516 of the gB amino acid sequence of HSV-1 can be found in amino acid position 425 of the unprocessed gB of HHV6 and amino acid position 403 of the processed gB of HHV6. The amino acid sequence of the unprocessed gB of HHV6 is depicted in SEQ ID NO:25. The amino acid sequence of the mature gB of HHV6 is depicted in SEQ ID NO:26. A modified immature gB of HHV6 carrying a Tyr425Pro substitution is depicted in SEQ ID NO:27. The corresponding mature form of the protein, including the same substitution in position 403, is depicted in SEQ ID NO:28.

Further, the residue corresponding to the amino acid in position 516 of the gB amino acid sequence of HSV-1 can be found in amino acid position 421 of the unprocessed gB of HHV7 and amino acid position 399 of the processed gB of HHV7. The amino acid sequence of the unprocessed gB of HHV7 is depicted in SEQ ID NO:29. The amino acid sequence of the mature gB of HHV7 is depicted in SEQ ID NO:30. A modified immature gB of HHV7 carrying a His421Pro substitution is depicted in SEQ ID NO:31. The corresponding mature form of the protein, including the same substitution in position 399, is depicted in SEQ ID NO:32.

Further, the residue corresponding to the amino acid in position 516 of the gB amino acid sequence of HSV-1 can be found in amino acid position 471 of the unprocessed gB of HHV8 and amino acid position 445 of the processed gB of HHV8. The amino acid sequence of the unprocessed gB of HHV8 is depicted in SEQ ID NO:33. The amino acid sequence of the mature gB of HHV8 is depicted in SEQ ID NO:34. A modified immature gB of HHV8 carrying a Gly471Pro substitution is depicted in SEQ ID NO:35. The corresponding mature form of the protein, including the same substitution in position 445, is depicted in SEQ ID NO:36.

Accordingly, in one embodiment of the invention the modified herpesvirus envelope gB of the invention comprises
(a) the amino acid sequence of any of SEQ ID NO:11, 12, 15, 16, 23, 24, 27, 28, 31, 32, 35 or 36;
(b) an amino acid sequence which has at least 90% identity to the amino acid sequence of SEQ ID NO:11, 12, 15, 16, 23, 24, 27, 28, 31, 32, 35 or 36;
(c) an immunologically active fragment of any of (a) or (b).

In a particularly preferred embodiment, the modified herpesvirus envelope gB comprises or consists of an amino acid sequence selected from the group of SEQ ID NO:11, 12, 15, 16, 23, 24, 27, 28, 31, 32, 35 or 36.

In addition, it has been found in the course of the invention that another particularly suitable modified gB which is locked to the prefusion confirmation can be obtained by mutation of the Ser residue in the position corresponding to position 392 of the unprocessed HSV-1 envelope gB and simultaneous mutation of the Gln residue in the position corresponding to position 532 of the unprocessed HSV-1 envelope gB. The Ser residue is located in position 362 and the Gln residue in position 502 of the mature, processed HSV-1 envelope gB set forth in SEQ ID NO:2. While these two residues are located in different domains, they are in close proximity in the prefusion conformation. The structural reorganisation during fusion means the two domains will end up in different locations in the postfusion conformation.

Preferably, the amino acids located in positions corresponding to position 392 and 532 of the unprocessed HSV-1 envelope gB are both replaced by a Cys residue. The Cys residues form a disulphide bond that holds the two domains in close proximity. As shown in the below examples, when gB carrying the two mutations Ser392Cys and Gln532Cys is overexpressed in cells, extracellular vesicles are formed with the protein stabilised in pre-fusion conformation on their surface.

A modified immature gB of HSV-1 carrying both the Ser392Cys and the Gln532Cys substitution is depicted in SEQ ID NO:5. The corresponding mature form of the protein, including the same two substitutions, is depicted in SEQ ID NO:6. In one embodiment, the modified herpesvirus envelope gB of the invention comprises
(a) the amino acid sequence of SEQ ID NO:5 or SEQ ID NO:6;
(b) an amino acid sequence which has at least 90% identity to the amino acid sequence of SEQ ID NO:5 or SEQ ID NO:6;
(c) an immunologically active fragment of any of (a) or (b).

In a particularly preferred embodiment, the modified herpesvirus envelope gB comprises or consists of the amino acid sequence of SEQ ID NO:5. In yet another particularly preferred embodiment, the modified herpesvirus envelope gB comprises or consists of the amino acid sequence of SEQ ID NO:6.

The invention of course also refers to a modified gB which includes all of the above-discussed mutations, i.e. the helix-breaking mutation of the His residue in the position corresponding to position 516 of the unprocessed HSV-1 envelope gB as well as the two mutations of the Ser residue in the position corresponding to position 392 and the mutation of the Gln residue in the position corresponding to position 532 of the unprocessed HSV-1 envelope gB.

A modified immature gB of HSV-1 carrying all three mutations is depicted in SEQ ID NO:7. The corresponding mature form of the protein, including the same three substitutions, is depicted in SEQ ID NO:8. In one embodiment, the modified herpesvirus envelope gB of the invention comprises
(a) the amino acid sequence of SEQ ID NO:7 or SEQ ID NO:8;
(b) an amino acid sequence which has at least 90% identity to the amino acid sequence of SEQ ID NO:7 or SEQ ID NO:8;
(c) an immunologically active fragment of any of (a) or (b).

In a particularly preferred embodiment, the modified herpesvirus envelope gB comprises or consists of the amino acid sequence of SEQ ID NO:7. In yet another particularly preferred embodiment, the modified herpesvirus envelope gB comprises or consists of the amino acid sequence of SEQ ID NO:8.

The Ser residue in position 392 and the Gln residue in position 532 of the unprocessed gB of HSV-1 are also conserved in the corresponding gB proteins of other herpesviruses. For example, these residues correspond to the Ser in position 397 and the Gln in position 543 of the unprocessed gB of VZV, respectively. In the mature gB of VZV, these residues are located in positions 326 and 472, respectively.

A modified immature gB of VZV carrying both the Ser397Cys and the Gln543Cys substitution is depicted in SEQ ID NO:17. The corresponding mature form of the protein, including the same two substitutions, is depicted in SEQ ID NO:18. In one embodiment, the modified herpesvirus envelope gB of the invention comprises
(a) the amino acid sequence of SEQ ID NO:17 or SEQ ID NO:18;
(b) an amino acid sequence which has at least 90% identity to the amino acid sequence of SEQ ID NO:17 or SEQ ID NO:18;
(c) an immunologically active fragment of any of (a) or (b).

In a particularly preferred embodiment, the modified herpesvirus envelope gB comprises or consists of the amino acid sequence of SEQ ID NO:17. In yet another particularly preferred embodiment, the modified herpesvirus envelope gB comprises or consists of the amino acid sequence of SEQ ID NO:18.

The invention also relates to a modified gB of VZV which includes all of the above mutations, i.e. the helix-breaking mutation of the His residue in position 527 of the unprocessed gB of VZV as well as the two mutations of the Ser residue in the position 397 and the Gln in position 543 of the unprocessed gB of VZV.

A modified immature gB of VZV carrying all three mutations is depicted in SEQ ID NO:19. The corresponding mature form of the protein, including the same three substitutions, is depicted in SEQ ID NO:20. In one embodiment, the modified herpesvirus envelope gB of the invention comprises
(a) the amino acid sequence of SEQ ID NO:19 or SEQ ID NO:20;
(b) an amino acid sequence which has at least 90% identity to the amino acid sequence of SEQ ID NO:19 or SEQ ID NO:20;
(c) an immunologically active fragment of any of (a) or (b).

In a particularly preferred embodiment, the modified herpesvirus envelope gB comprises or consists of the amino acid sequence of SEQ ID NO:19. In yet another particularly preferred embodiment, the modified herpesvirus envelope gB comprises or consists of the amino acid sequence of SEQ ID NO:20.

The above mutations in the gB protein lock the gB in the prefusion conformation, thereby inhibiting infection of the cell with the herpesvirus. Preferably, infection of a cell with a herpesvirus expressing the modified gB of the invention is inhibited by at least 70%, more preferably at least 75%, at least 80%, at least 85%, at least 90%, at least 95% or more as compared to infection with the corresponding wild-type herpesvirus. For example, if the mutated gB is expressed by a genetically modified HSV-1, infection of a cell with said genetically modified HSV-1 will be inhibited by at least 70%, more preferably at least 75%, at least 80%, at least 85%, at least 90%, at least 95% or more as compared to the corresponding wild-type HSV-1. The inhibition of infection can be determined by common methods, such as plaque reduction assays.

Any herpesvirus gB can be used for modification in accordance with the present invention. However, it is preferred that the gB is derived from a human herpesvirus, i.e. a herpesvirus selected from the group of Herpes Simplex Virus 1 and 2 (HSV-1, HSV-2), Varicella-Zoster Virus (VZV), Epstein—Barr virus (EBV), Human Cytomegalovirus (HCMV), Human Herpesvirus 6A and 6B (HHV-6A, HHV-6B), Human Herpesvirus 7 (HHV-7), and Kaposi's Sarcoma-associated Herpesvirus (KSHV). In a particularly preferred embodiment, the modified gB is derived by the modification of human HSV-1. In another particularly preferred embodiment, the modified gB is derived by the modification of human HSV-2. In yet another particularly preferred embodiment, the modified gB is derived by the modification of human VZV. In the case of VZV, the His residues that corresponds to the His in position 516 in the gB of HSV-1 is located in position 527.

In addition, it has been found in the course of the invention that another particularly suitable modified gB which produces a higher amount of extracellular vesicles can be obtained by mutation of the C-terminally located endocytosis motif (Niazy et al., 2017). The endocytosis motif consists of four residues: Tyr-X-X-Z (X=any residue, Z=hydrophobic residue) and causes the protein to be re-internalised into the cell from the plasma membrane. A single point mutation in this endocytosis motif changing the Tyr to Ala has the same effect. Therefore, in order to increase the vesicle yield, the Tyr to Ala mutation can be introduced. This motif is conserved in many Herpesvirus gB proteins. In HSV-1 the Tyr residue to be mutated is in position 889. In VZV the corresponding tyrosine residue is in position 920.

As indicated above, it is preferred that the modified gB protein of the invention comprises or consists of the amino acid sequence of SEQ ID NO:3, SEQ ID NO:4, SEQ ID NO:5, SEQ ID NO:6, SEQ ID NO:7, SEQ ID NO:8, SEQ ID NO:11, SEQ ID NO:12, SEQ ID NO:15, SEQ ID NO:16, SEQ ID NO:17, SEQ ID NO:18, SEQ ID NO:19, SEQ ID NO:20, SEQ ID NO:23 SEQ ID NO:24, SEQ ID NO:27, SEQ ID NO:28, SEQ ID NO:31, SEQ ID NO:32, SEQ ID NO:35 or SEQ ID NO:36.

Alternatively, the modified gB protein of the invention comprises or consists of an amino acid sequence which has at least 90% identity to one of the amino acid sequences of SEQ ID NO:3, SEQ ID NO:4, SEQ ID NO:5, SEQ ID NO:6, SEQ ID NO:7, SEQ ID NO:8, SEQ ID NO:11, SEQ ID NO:12, SEQ ID NO:15, SEQ ID NO:16, SEQ ID NO:17, SEQ ID NO:18, SEQ ID NO:19, SEQ ID NO:20, SEQ ID NO:23 SEQ ID NO:24, SEQ ID NO:27, SEQ ID NO:28, SEQ ID NO:31, SEQ ID NO:32, SEQ ID NO:35 or SEQ ID NO:36. Preferably, the modified gB protein of the invention comprises or consists of an amino acid sequence which has a sequence identity of 91%, 92%, 93%, 94%, 95%, 96%, 97%, 98%, or 99% to the above reference sequences, provided that the sequences also comprise the mutations which are encompassed by the reference sequences. Preferably, the sequence identity is determined over a length of at least 100 amino acids, more preferably at least 150 amino acids, at least 200 amino acids, at least 250 amino acids, at least 300 amino acids, at least 350 amino acids, at least 400 amino acids, at least 450 amino acids, at least 500 amino acids, at least 550 amino acids, at least 600 amino acids, at least 650 amino acids, at least 700 amino acids, at least 750 amino acids, at least 800 amino acids, at least 850 amino acids, or at least 900 amino acids.

In one embodiment, the modified gB of the invention shares a sequence identity with an amino acid sequence of any of SEQ ID NOs:3-8, SEQ ID NO:11-12, SEQ ID NO:15-20, SEQ ID NO:23-24, SEQ ID NO:27-28, SEQ ID NO:31-32, or SEQ ID NO:35-36 of at least 95% over a length of at least 100 amino acids, at least 200 amino acids, at least 300 amino acids, at least 400 amino acids, at least 500 amino acids, at least 600 amino acids, at least 700 amino acids, at least 800 amino acids, or at least 900 amino acids. It is particularly preferred that the sequence identity is at least 95% over the full length of the protein. In one embodiment, the modified gB of the invention shares a sequence identity with an amino acid sequence of any of SEQ ID NOs:3-8 of at least 96% over a length of at least 100 amino acids, at least 200 amino acids, at least 300 amino acids, at least 400 amino acids, at least 500 amino acids, at least 600 amino acids, at least 700 amino acids, at least 800 amino acids, or at least 900 amino acids. It is particularly preferred that the sequence identity is at least 96% over the full length of the protein. In one embodiment, the modified gB of the invention shares a sequence identity with an amino acid sequence of any of SEQ ID NOs:3-8, SEQ ID NO:11-12, SEQ ID NO:15-20, SEQ ID NO:23-24, SEQ ID NO:27-28, SEQ ID NO:31-32, or SEQ ID NO:35-36 of at least 97% over a length of at least 100 amino acids, at least 200 amino acids, at least 300 amino acids, at least 400 amino acids, at least 500 amino acids, at least 600 amino acids, at least 700 amino acids, at least 800 amino acids, or at least 900 amino acids. It is particularly preferred that the sequence identity is at least 97% over the full length of the protein. In one embodiment, the modified gB of the invention shares a sequence identity with an amino acid sequence of any of SEQ ID NOs:3-8, SEQ ID NO:11-12, SEQ ID NO:15-20, SEQ ID NO:23-24, SEQ ID NO:27-28, SEQ ID NO:31-32, or SEQ ID NO:35-36 of at least 98% over a length of at least 100 amino acids, at least 200 amino acids, at least 300 amino acids, at least 400 amino acids, at least 500 amino acids, at least 600 amino acids, at least 700 amino acids, at least 800 amino acids, or at least 900 amino acids. It is particularly preferred that the sequence identity is at least 98% over the full length of the protein. In one embodiment, the modified gB of the invention shares a sequence identity with an amino acid sequence of any of SEQ ID NOs:3-8, SEQ ID NO:11-12, SEQ ID NO:15-20, SEQ ID NO:23-24, SEQ ID NO:27-28, SEQ ID NO:31-32, or SEQ ID NO:35-36 of at least 99% over a length of at least 100 amino acids, at least 200 amino acids, at least 300 amino acids, at least 400 amino acids, at least 500 amino acids, at least 600 amino acids, at least 700 amino acids, at least 800 amino acids, or at least 900 amino acids. It is particularly preferred that the sequence identity is at least 99% over the full length of the protein.

In order to determine the sequence identity between two amino acid sequences, these sequences are usually aligned for optimal comparison. For example, gaps can be introduced in the sequence of a first amino acid sequence for optimal alignment with a second amino acid sequence. The amino acids at corresponding positions are then compared. If identical amino acids occur in corresponding positions in the first and second amino acid sequence, the sequences are identical at that position. A percentage sequence identity between two amino acid sequences means that, when aligned, the recited percentage of amino acids are identical in comparing both sequences. A percentage sequence identity can be determined by using software programs that are widely known in the art, for example the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

The present invention also provides a nucleic acid encoding a mutated gB as described hereinabove. A plasmid comprising such a nucleic acid is also provided. As used herein, a plasmid refers to an extrachromosomal circular DNA capable of autonomous replication in a cell.

In another aspect, the invention pertains to a herpesvirus or a recombinant herpesvirus vector that comprises a modified gB as described herein or a nucleotide sequence encoding the same. As used herein, the term “herpesvirus” refers to a virus or virus particle that can be categorized as a herpesvirus, including all types and subtypes that occur naturally or have been recombinantly produced. In contrast, a “viral vector” refers to a virus or viral particle that comprises a polynucleotide, which is exogenous to the viral genome, such as a transgene, and which is to be delivered to a host cell by in vivo, ex vivo or in vitro methods. The herpesvirus or herpesvirus vector of the invention is preferably derived from a herpesvirus of the Herpesviridae family, and more preferably from a herpesvirus of the genus Simplexvirus. In a most preferred embodiment, the herpesvirus or herpesvirus vector is or is derived from HSV-1 or HSV-2.

In another preferred embodiment, the herpesvirus or herpesvirus vector of the invention comprises, as part of its genome, a nucleotide sequence encoding the modified gB as defined above. It is particularly preferred that this herpesvirus or herpesvirus vector only comprises a gene encoding the mutated gB, but not any other gene encoding the non-mutated version of gB. This ensures that the herpesvirus or herpesvirus vector is unable to infect the target cells.

In yet another aspect, the invention relates to the use of a modified gB as described herein, preferably a recombinantly produced gB, a nucleotide sequence or plasmid encoding the same, or a herpesvirus or a recombinant herpesvirus vector that comprises said modified gB and/or nucleotide sequence in medicine, i.e. for therapeutic purposes. Since the mutated herpesvirus gB prevents infection, the protein or a nucleotide sequence or plasmid encoding the same, as well as a virus or vector expressing said protein are useful for treating or preventing herpesvirus infections. Accordingly, the invention particularly relates to a modified gB, preferably a recombinantly produced gB, as described herein, a nucleotide sequence or plasmid encoding the same or a herpesvirus or a recombinant herpesvirus vector that comprises said modified gB or nucleotide sequence for use in a method of treating or preventing a herpesvirus infection in a subject. The subject preferably is a mammalian subject, more preferably a human subject. The subject preferably suffers from infection with a herpesvirus, preferably a herpesvirus that infects humans, such as HSV-1 or HSV-2.

In yet another aspect, the invention refers to the mutated herpesvirus gB, preferably a recombinantly produced gB, a nucleotide sequence or plasmid encoding the same, or a herpesvirus or a recombinant herpesvirus vector that comprises said modified gB and/or nucleotide sequence or plasmid for use in a method of vaccinating a subject. The subject preferably is a mammalian subject, more preferably a human subject. The vaccination shall protect the subject against herpesvirus infection, preferably a herpesvirus infection caused by a virus selected from the group consisting of HSV-1, HSV-2, VZV, EBV, HCMV, HHV-6A, HHV-6B, HHV-7 and KSHV. The vaccination shall also protect the subject against a disease that is caused by an infection with any of the above viruses, e.g. cold sore, genital herpes, herpesviral encephalitis and keratitis, neonatal herpes, chickenpox, shingles, infectious mononucleosis, Burkitt lymphoma, Hodgkin's lymphoma, cytomegalic inclusion disease, cerebral calcification after congential HCMV infection, exanthema subitem, Kaposi's sarcoma or primary effusion lymphoma.

The vaccination is preferably carried out with the modified herpesvirus gB of the respective virus, preferably a recombinantly produced gB protein, a nucleotide sequence or plasmid encoding the same, or a herpesvirus or a recombinant herpesvirus vector that comprises said modified gB or the nucleotide sequence or plasmid.

The invention also provides a cell that comprises a herpesvirus gB as described herein, a nucleotide sequence or plasmid encoding the same, or a herpesvirus or recombinant herpesvirus vector that comprises said modified gB and/or nucleotide sequence or plasmid. The cell can be any eukaryotic cell, but it will preferably be a mammalian cell, and more preferably a human cell. Preferably, the invention provides a cell that is transfected with a nucleotide sequence encoding a modified herpesvirus gB as described herein or a herpesvirus or recombinant herpesvirus vector which expresses the mutated herpesvirus gB of the invention.

In a further aspect, the invention provides a pharmaceutical composition or vaccine comprising a modified gB as described herein, preferably a recombinantly produced gB protein, a nucleotide sequence or plasmid encoding the same or a herpesvirus or a recombinant herpesvirus vector that comprises said modified gB and/or nucleotide sequence or plasmid. Preferably, the invention provides a pharmaceutical composition or vaccine comprising a herpesvirus or a herpesvirus vector of the present invention that expresses a mutated gB as defined herein. The pharmaceutical composition can be formulated for various routes of administration. For example, the composition can be formulated for oral administration in the form of a capsule, a liquid or the like. However, it is preferred that the pharmaceutical composition or vaccine is administered parenterally, preferably by intravenous injection or intravenous infusion. Compositions, which are suitable for administration by injection and/or infusion typically include solutions and dispersions, and powders from which corresponding solutions and dispersions can be prepared. Such compositions will comprise a mutated protein, nucleic acid, herpesvirus or herpesvirus vector as defined hereinabove and at least one pharmaceutically acceptable carrier. Suitable pharmaceutically acceptable carriers for intravenous administration include bacteriostatic water, Ringer's solution, physiological saline, phosphate buffered saline (PBS) and Cremophor EL™. Sterile compositions for the injection and/or infusion can be prepared by introducing the mutated protein, nucleic acid, herpesvirus or herpesvirus vector as defined hereinabove in the required amount into an appropriate carrier, and then sterilizing by filtration. Compositions for administration by injection or infusion should remain stable under storage conditions after their preparation over an extended period of time. The compositions can contain a preservative for this purpose. Suitable preservatives include chlorobutanol, phenol, ascorbic acid and thimerosal. The preparation of corresponding formulations and suitable adjuvants is described, for example, in “Remington: The Science and Practice of Pharmacy,” Lippincott Williams & Wilkins; 21^{st} edition (2005).

The pharmaceutical composition will comprise the modified gB as described herein, preferably a recombinantly produced gB, or the nucleotide sequence or plasmid encoding same, or the herpesvirus or herpesvirus vector in a therapeutically effective amount, i.e., in an amount that is sufficient for improving at least one symptom of the disease to be treated to the patient or to prevent the progression of the disease to the patient. A therapeutically effective amount of the modified gB protein, or the nucleotide sequence or plasmid encoding same, or the herpesvirus or herpesvirus vector causes a positive change in at least one of the symptoms, i.e., a change, which results in the phenotype of the affected subject approximating the phenotype of a healthy subject who does not suffer from the respective disease. In one preferred embodiment, the administration of the modified gB described herein, or the nucleotide sequence or plasmid encoding same, or the herpesvirus or herpesvirus vector occurs in an amount, which leads to a complete or substantially complete healing of the disease or dysfunction to be treated. A therapeutically effective amount will generally be non-toxic for the subject who undergoes the treatment.

The exact amount of the protein, nucleotide sequence, plasmid, herpesvirus or herpesvirus vector which must be administered to achieve a therapeutic effect depends on several parameters. Factors that are relevant to the amount of the herpesvirus or herpesvirus vector to be administered are, for example, the route of administration, the nature and severity of the disease, the disease history of the patient being treated, as well as the age, weight, height, and health of the patient. A therapeutically effective amount of the protein, nucleotide sequence, plasmid, herpesvirus or herpesvirus vector can be determined by a person skilled in the art on the basis of general knowledge and the present disclosure.

If a herpesvirus or herpesvirus vector is used as a vaccine or therapeutic agent, the amount of said herpesvirus or vector to be administered preferably corresponds to a dose in the range of 1.0×10¹⁰ to 1.0×10¹⁴ vg/kg (virus genomes per kg body weight), although a range of 1.0×10¹¹ to 1.0×10¹³ vg/kg is more preferred, and a range of 5.0×10¹¹ to 5.0×10¹² vg/kg is still more preferred, and a range of 1.0×10¹² to 5.0×10¹² is still more preferred. A dose of about 2.5×10¹² vg/kg is most preferred.

When formulated as a vaccine, the above components, i.e. the modified gB, the nucleotide sequence or plasmid encoding same, or the herpesvirus or recombinant herpesvirus vector, will be admixed with an adjuvant. An adjuvant is a compound that enhances the immune responses in a subject to whom the vaccine is administered. Adjuvants, which are commonly used for the preparation of vaccines include, but are not limited to, mineral salts, such as aluminium salts or calcium salts; oil-in-water emulsions; saponin compounds, such as QS7, QS17, QS18, or QS21; immunostimulatory oligonucleotides, such as oligonucleotides sequences containing a CpG motif; biodegradable microparticles, such as particles of poly-α-hydroxy acid, polyhydroxybutyric acid, polyorthoester, or the like; liposomes; muramyl peptides; and the like. According to a preferred embodiment of the present invention, the adjuvant used is an aluminium salt, in particular aluminium hydroxide.

In yet another aspect, the invention relates to the use of a modified herpesvirus gB as described herein, a nucleotide sequence or plasmid encoding the same, or a herpesvirus or recombinant herpesvirus vector as described herein for the preparation of a vaccine. This vaccine is preferably effective against a disease that is caused by herpesvirus infection selected from the group consisting of HSV-1, HSV-2, VZV, EBV, HCMV, HHV-6A, HHV-6B, HHV-7 and KSHV. The vaccine therefore protects against a disease caused by any of these viruses, including cold sore, genital herpes, herpesviral encephalitis and keratitis, neonatal herpes, chickenpox, shingles, infectious mononucleosis, Burkitt lymphoma, Hodgkin's lymphoma, cytomegalic inclusion disease, cerebral calcification after congential HCMV infection, exanthema subitem, Kaposi's sarcoma or primary effusion lymphoma.

### BRIEF DESCRIPTION OF THE FIGURES

FIG. 1 shows the results from an SDS-polyacrylamide gel electrophoresis (PAGE) and Coomassie stain of extracellular vesicle purifications, obtained from cells transfected with the HSV-1 gB wild type (wt) and single-point mutants, and Western blot analysis of the cell lysate and extracellular vesicles. Mature gB protein is marked by a black arrowhead.

FIG. 2 shows the fusion activity of gB wt, a fusion-null construct, or a single-point mutant in combination with gH/gL and gD in a cell-cell fusion assay. Activities are normalized to wt fusion activity level.

FIG. 3 shows the analysis of purified extracellular vesicles formed by WT and gB His516Pro using cryo-EM. The long, postfusion form of gB is indicated by blue lines, and the short form is indicated by orange lines. Lower left images show the top views of gB trimers. Scale bars: 25 nm.

FIG. 4 shows the size distribution of vesicles found with short and long gB form on vesicles formed by wt gB (n=183) and gB His516Pro (n=114). Boxes indicate range from first to third quartile with whiskers showing ±1.5 interquartile range and outliers marked as points. *no range given, as only two vesicles were found displaying the long form of gB His516Pro. The red line marks the median diameter.

FIG. 5 shows the results from an SDS-polyacrylamide gel electrophoresis (PAGE) and Coomassie stain of extracellular vesicle purifications, obtained from cells transfected with the HSV-1 gB wild type (wt), a truncated construct missing the last 36 aa (868t) and a single-point mutant changing amino acid 898 from Tyrosine to Alanine.

FIG. 6 shows the Cryo-ET slices of gB mutant vesicles from different Herpesvirus species—HSV-1 and VZV. The gB protein is stabilised in its pre-fusion conformation by the corresponding mutations H516P and H527P.

FIG. 7 shows Cryo-ET slices of gB mutant vesicles. The gB protein is stabilised by the introduced disulphide bond or the disulphide bond combined with the previously described proline mutation in its pre-fusion conformation.

### EXAMPLES

The present invention is further described in more detail by the following examples which are only provided for illustrating the invention and which are not to be construed as limiting the scope of the invention. The following material and methods were used in the Examples.

### Example 1: Expression Plasmid Construction and Vesicle Preparation

The sequence for a 5xGS linker was added to the C terminus of the gB gene, followed by a 6xHis tag in the pEP98 plasmid. Single-point mutations were created using the Agilent QuikChange II Kit or NEB Q5 kit for site-directed mutagenesis.

Vesicles were subsequently prepared as described (Zeev-Ben-Mordehai et al. (2014). In brief, BHK-21 cells were grown in GMEM (Glasgow's Minimal Essential Medium) supplemented with 20 mM Hepes (pH 7.4), 2% (v/v) TPB (tryptose phosphate broth), and 2% (v/v) fetal bovine serum. At around 70% confluency, cells were transiently transfected. Cells were grown for an additional 48 hours with a media exchange to serum-free GMEM after 24 hours. Vesicles were harvested from the supernatant by differential centrifugation and resuspended in 20 mM Hepes (pH 8) and 150 mM NaCl.

Vesicle preparations were tested in SDS—polyacrylamide gel electrophoresis (PAGE) followed by Coomassie staining or Western blotting with a rabbit anti-His6 antibody (Abcam) followed by anti-rabbit horseradish peroxidase (HRP) (Sigma-Aldrich Chemie GmbH). After supernatants were removed for vesicle preparations, cells were washed with cold phosphate-buffered saline (PBS) and detached using cell scrapers. Cells were pelleted by centrifugation (5 min, 4500 g, 4° C.), transferred into 1.5-ml tubes, and washed in cold PBS again before resuspension in radioimmunoprecipitation buffer, 100 μl per T175 flask [50 mM Tris (pH 8), 1% NP-40, 0.1% SDS, 150 mM NaCl, 0.5% sodium deoxycholate, 5 mM EDTA, 1 mM phenylmethylsulfonyl fluoride]. Samples were shaken at 4° C. for 30 min before being spun at 500 g for 10 min. Supernatants were mixed in SDS sample buffer and run in parallel with vesicle samples in SDS-PAGE. For loading control, Western blots were re-probed using a mouse anti-glyceraldehyde-3-phosphate dehydrogenase antibody (Sigma-Aldrich Chemie GmbH), followed by anti-mouse HRP (Sigma-Aldrich Chemie GmbH).

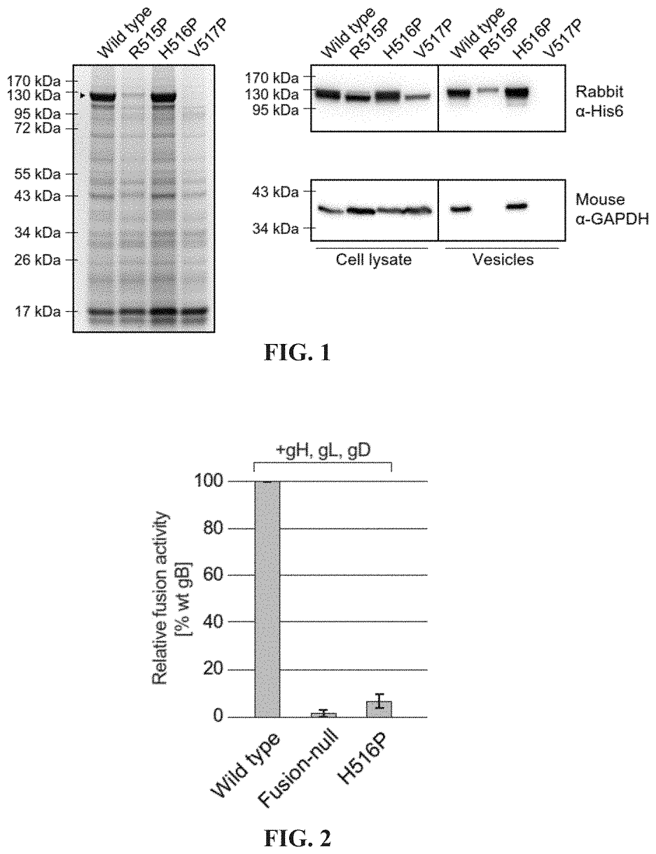

Results: Helix-breaking point mutations to proline were introduced individually at residues 515 to 517 of gB. It can be seen in FIG. 1 that a wild type (WT)-like expression of full-length mutant gB was obtained only with a proline at position 516. Mutations in adjacent positions resulted in low or no expression of the mature protein and absence of vesicles. The amount and ratio of fully to partly glycosylated gB on extracellular vesicles, detectable as a single or double band, respectively (Sommer & Courtney, 1991), were not altered by the His516Pro mutation, suggesting that expression and posttranslational modifications were not affected (FIG. 1).

### Example 2: Transient Transfection-Based Cell-Cell Fusion Assay

Fusion activity of the different HSV-1 gB constructs was determined after transient transfection of RK13 cells as described (Vallbracht, et al., 2017a). Briefly, cells were transfected with 200 ng each of the expression plasmids for enhanced green fluorescent protein (EGFP) (pEGFP-N1; Clontech), nectin-1, and HSV-1 glycoproteins gD, gL, gH, and gB or mutant gB in 100 μl of Opti-MEM using 1 μl of Lipofectamine 2000. Twenty-four hours after transfection, the cells were fixed with 3% paraformaldehyde and analyzed using an Eclipse Ti-S fluorescence microscope and NIS-Elements Imaging Software (Nikon). Fusion activity was determined by multiplication of the number of syncytia by the mean syncytia area within 10 fields of view (5.5 mm² each). Each experiment was repeated four times, and average percent values of positive control transfections as well as standard deviations were calculated.

Results: When testing the fusion activity of gB His516Pro in a cell-cell fusion assay (Vallbracht, et al. 2017b) in the presence of gD, gH, and gL. All four glycoproteins are essential and sufficient for HSV-1 membrane fusion and entry (Davis-Poynter et al., 1994; Rogalin & Heldwein, 2016). Compared to wt gB, the cell-cell fusion activity of gB His516Pro was reduced to 6.5% (SD: 3.2%) (FIG. 2), only slightly above the background level [1.6% (SD: 1.4%)] of a fusion-null construct (Cai et al., 1988; Chowdary & Heldwein, 2010). This fusion inhibition is consistent with a block in the pre- to postfusion state transition, functionally locking the protein.

### Example 3: Electron Cryomicroscopy (Cryo-EM) to Determine Vesicle Size Distribution

It has been reported that wt gB is present in two major forms on extracellular vesicles (Zeev-Ben-Mordehai et al., 2016): an extended one (of about 16 nm in length) corresponding to the postfusion conformation and mostly found on small vesicles (approximate median diameter=59 nm) and a compact one (of about 12 nm in length) corresponding to the prefusion state, the latter of which is found on larger vesicles (approximate median diameter=98 nm). For wt gB, about 30% of vesicles predominantly presented the extended form, while 70% predominantly presented the compact form (n=183), similar to previous observations (Zeev-Ben-Mordehai et al., 2016).

For grid preparation, 3.5 μl of vesicles were mixed with 10-nm gold fiducials on Quantifoil R2/1 grids and plunge-frozen in a propane/ethane mixture using a manual plunge freezer. Microscopy was performed using a Tecnai F30 “Polara” microscope (FEI Thermo Fisher Scientific) at 300 kV equipped with a Quantum 964 post-column energy filter (Gatan) operated in zero-loss imaging mode. Images were recorded on a 4k×4k K2 Summit electron detector with a calibrated pixel size of 0.14 nm at the specimen level. Transmission images were recorded using SerialEM (49) at a −3-μm defocus. Vesicle diameters were measured in 3dmod.

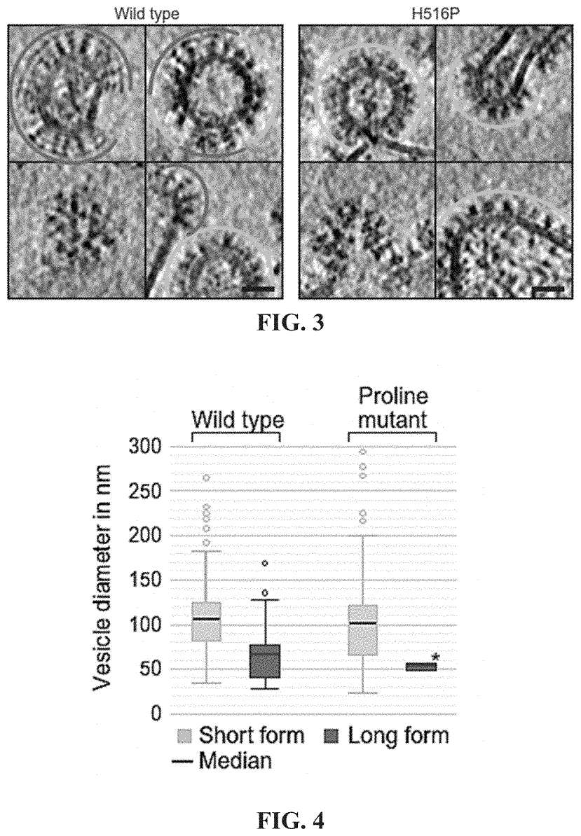

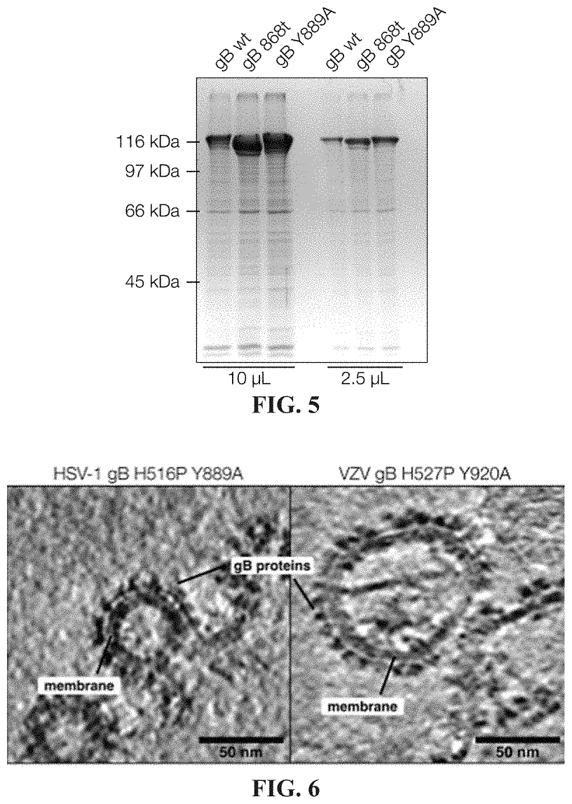

Results: The results of the cryo-EM are depicted in FIG. 3. For gB His516Pro, total vesicle production was similar to wt gB (FIG. 1), but the ratio was drastically shifted to 98.2% (n=112) of vesicles displaying solely the compact form and only 1.8% displaying the extended form (n=2), with comparable size distributions as the wt vesicles (approximate median diameter=53 nm for extended form; approximate median diameter=90 nm for compact form) (FIG. 4). Thus, introducing the His516Pro mutation apparently inhibited the transition of gB from the compact to the extended form. The small number of vesicles displaying the extended form of gB His516Pro parallels the remaining residual fusion activity, which is marginally above background levels (FIG. 2).

### Example 4: Recombinant Expression and Crystallization of Modified gB

The synthetic gene encoding residues 31 to 730 of HSV-1 gB including the His516Pro mutation was codon-optimized for protein expression in insect cells and cloned into the pT350 vector (Krey et al., 2010) between the vector-encoded Bip signal peptide that drives protein secretion and a double strep tag. The gB modified ectodomain was produced in S2 Drosophila cells using standard methods (Backovic & Krey, 2016). The protein was purified on a Strep-Tactin affinity resin and by size exclusion chromatography using Superdex 200 16/60 column and 10 mM Tris (pH 8) and 50 mM NaCl as running buffer. The protein was concentrated to 6.4 mg/ml and crystallized in 0.1 M Tris (pH 8), 18% ethanol at the Institut Pasteur core facility for crystallization (Weber et al., 2019). They were flash-frozen in liquid nitrogen in cryosolution containing 0.1 M Tris (pH 8), 20% ethanol.

Results: The results of the crystallisation followed by X-ray diffraction and density map acquisition showed that the ectodomain comprising the single His516Pro mutation and lacking the transmembrane region still adopts the postfusion conformation. Therefore, a stabilisation of the prefusion conformation by the His516Pro mutation is only achieved in combination with a full-length gB protein (aa 1-904), natively embedded in a membrane.

### Example 5: Vesicle Production Test

SDS-PAGE analysis (Coomassie stained) of HSV-1 vesicles from BHK-21 cells transfected with either wild-type gB, a truncated construct ending at residue 868 or gB with a Y889A point mutation. Vesicles were harvested 48 h post transfection. Vesicle samples were mixed in a 2:1 ratio with SDS sample buffer and were loaded in two volumes— 10 μL on the left, 2.5 μL on the right.

Results: It was found that removal or mutation of the c-terminal endocytosis motif increases the yield of vesicles that can be purified from the supernatant of transfected cells.

### Example 6: Cryo-EM to Determine gB Conformation on the Vesicle Surfaces

Vesicles were produced as and prepared as described (Zeev-Ben-Mordehai et al., 2014) using plasmids encoding gB of HSV-1 or VZV and comprising two kinds of mutations to stabilise the gB protein in prefusion conformation either alone or in combination. In addition, the gB genes all contained an additional mutation in the C-terminal Tyr-X-X-Z endocytosis motif that leads to an increased release of gB containing vesicles. Vesicles were analysed by cryo-EM after plunge freezing on grids (see Example 3). Tomographic imaging was done using a Titan Krios microscope (Thermo Fisher Scientific) at 300 kV with a 70 μm C2 aperture and post-column Quantum energy filter operated in zero-loss mode using an energy slit of 20 eV and K2 Summit direct electron detector in counting mode (Gatan). SerialEM was used for automated data collection. IMOD software suite was used to reconstruct the tomograms.

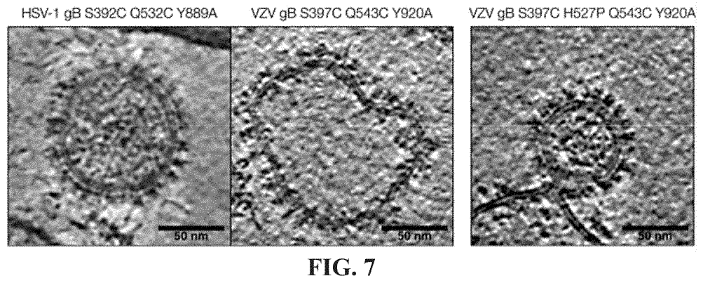

Results: Example images (slices of tomograms) of vesicles displaying mutated forms of gB are shown in FIGS. 6 and 7. Introduction of the His516Pro mutation in HSV-1 gB stabilises the protein in pre-fusion conformation. The same effect is seen when the corresponding residue is mutated to proline in VZV gB (His527Pro) (FIG. 6). A similar stabilisation is achieved by introduction of two mutations S392C and Q532C in HSV-1 gB. By introduction of the same amino acid changes in the corresponding residues of VZV gB S397C and Q543C a similar stabilisation of the protein in prefusion conformation is achieved. The two mutations can also be combined with the helix breaking mutation for an additional stabilisation of the prefusion conformation (FIG. 7).

### LITERATURE

1. GBD Mortality and Causes of Death (2015), Global, regional, and national life expectancy, all-cause mortality, and cause-specific mortality for 249 causes of death, 1980-2015: a systematic analysis for the Global Burden of Disease Study, Lancet 388, 1459-1544 (2016).
2. M. J. Cannon, D. S. Schmid, T. B. Hyde (2010), Review of cytomegalovirus seroprevalence and demographic characteristics associated with infection. Rev Med Virol 20, 202-213.
3. E. G. Damato, C. W. Winnen (2002), Cytomegalovirus infection: perinatal implications. J

Obstet Gynecol Neonatal Nurs 31, 86-92.
4. C. James et al. (2020), Herpes simplex virus: global infection prevalence and incidence estimates, 2016. Bull World Health Organ 98, 315-329.
5. M. Zuhair et al. (2019), Estimation of the worldwide seroprevalence of cytomegalovirus: A systematic review and meta-analysis. Rev Med Virol 29, e2034.
6. M. Backovic, T. S. Jardetzky (2011), in Cell Fusion in Health and Disease II: Cell Fusion in Disease, vol. 714, pp. 91-101.
7. E. E. Heldwein et al. (2006), Crystal structure of glycoprotein B from herpes simplex virus 1.

Science 313, 217-220.
8. T. Zeev-Ben-Mordehai, (2016), Two distinct trimeric conformations of natively membrane-anchored full-length herpes simplex virus 1 glycoprotein B. Proc. Natl. Acad. Sci. U.S.A. 113, 4176-4181.
9. J. Fontana et al. (2017), The fusion loops of the initial prefusion conformation of herpes simplex virus 1 fusion protein point toward the membrane. mBio 8, e01268-17.
10. M. Patrone et al. (2014), Enhanced expression of full-length human cytomegalovirus fusion protein in non-swelling baculovirus-infected cells with a minimal fed-batch strategy. PLOS ONE 9, e90753.
11. T. Zeev-Ben-Mordehai et al. (2014), Extracellular vesicles: A platform for the structure determination of membrane proteins by cryo-EM. Structure 22, 1687-1692.
12. M. Sommer, R. J. Courtney (1991), Differential rates of processing and transport of herpes simplex virus type 1 glycoproteins gB and gC. J. Virol. 65, 520-525.
13. M. Vallbracht, et al. (2017a), Transient transfection-based fusion assay for viral proteins. Bio-protocol. 7, e2162.
14. M. Vallbracht, et al. (2017b), Functional relevance of the N-terminal domain of pseudorabies virus envelope glycoprotein H and its interaction with glycoprotein L. J. Virol. 91, e00061-17.
15. N. Davis-Poynter, et al. (1994), Analysis of the contributions of herpes simplex virus type 1 membrane proteins to the induction of cell-cell fusion. J. Virol. 68, 7586-7590.
16. H. B. Rogalin, E. E. Heldwein (2016), Characterization of vesicular stomatitis virus pseudotypes bearing essential entry glycoproteins gB, gD, gH, and gL of herpes simplex virus
1. J. Virol. 90, 10321-10328.
17. W. H. Cai et al. (1988), Role of glycoprotein B of herpes simplex virus type 1 in viral entry and cell fusion. J. Virol. 62, 2596-2604.
18. T. K. Chowdary, E. E. Heldwein (2010), Syncytial phenotype of C-terminally truncated herpes simplex virus type 1 gB is associated with diminished membrane interactions. J. Virol. 84, 4923-4935.
19. T. Krey, et al. (2010), The disulphide bonds in glycoprotein E2 of hepatitis C virus reveal the tertiary organization of the molecule. PLOS Pathog. 6, e1000762.
20. M. Backovic, T. Krey (2016), Stable Drosophila cell lines: An alternative approach to exogenous protein expression. Methods Mol. Biol. 1350, 349-358.
21. P. Weber et al. (2019), High-throughput crystallization pipeline at the Crystallography Core

Facility of the Institut Pasteur. Molecules 24, 4451.
22. S. L. Oliver et al. (2020), A glycoprotein B-neutralizing antibody structure at 2.8 Å uncovers a critical domain for herpesvirus fusion initiation, Nat Commun. August 11(1):4141.
23. N. Niazy et al. (2017), Misdirection of endosomal trafficking mediated by herpes simplex virus-encoded glycoprotein B, FASEB J, 31(4):1650-1667

1. A modified herpesvirus envelope glycoprotein-B, wherein the protein comprises a mutation in its amino acid sequence which stabilizes the prefusion confirmation of the protein by preventing the irreversible transition from the metastable prefusion to the postfusion conformation.

2. The modified herpesvirus envelope glycoprotein-B of claim 1, comprising a mutation of the residue in the position corresponding to position 516 of the unprocessed full-length Herpes simplex virus 1 (HSV-1) envelope glycoprotein-B set forth in SEQ ID NO:1.

3. The modified herpesvirus glycoprotein-B of claim 2, wherein the residue in the position corresponding to position 516 has been replaced by another amino acid residue.

4. The modified herpesvirus glycoprotein-B of claim 3, wherein the residue in the position corresponding to position 516 has been replaced by a proline residue.

5. The modified herpesvirus glycoprotein-B of claim 1, wherein the protein comprises or consists of
(a) the amino acid sequence of any of SEQ ID NO:3, 4, 11, 12, 15, 16, 23, 24, 27, 28, 31, 32, 35 or 36;
(b) an amino acid sequence which has at least 90% identity to the amino acid sequence of SEQ ID NO: 3, 4, 11, 12, 15, 16, 23, 24, 27, 28, 31, 32, 35 or 36;
(c) an immunologically active fragment of any of (a) or (b).

6. The modified herpesvirus envelope glycoprotein-B of claim 1, comprising a mutation of the Ser residue in the position corresponding to position 392 and a mutation of the Gln residue in the position corresponding to position 532 of the unprocessed full-length Herpes simplex virus 1 (HSV-1) envelope glycoprotein-B set forth in SEQ ID NO: 1.

7. The modified herpesvirus glycoprotein-B of claim 6, wherein the Ser residue in the position corresponding to position 392 and the Gln residue in the position corresponding to position 532 of the unprocessed full-length Herpes simplex virus 1 (HSV-1) envelope glycoprotein-B have been replaced by a Cys residue.

8. The modified herpesvirus glycoprotein-B of claim 6, wherein the protein comprises or consists of
(a) the amino acid sequence of SEQ ID NO: 5, 6, 17 or 18;
(b) an amino acid sequence which has at least 90% identity to the amino acid sequence of SEQ ID NO:5, 6, 17 or 18;
(c) an immunologically active fragment of any of (a) or (b).

9. The modified herpesvirus glycoprotein-B of claim 1, said protein comprising:
(a) a mutation of the His residue in the position corresponding to position 516 of the unprocessed full-length Herpes simplex virus 1 (HSV-1) envelope glycoprotein-B set forth in SEQ ID NO:1, and
(b) a mutation of the Ser residue in the position corresponding to position 392 and a mutation of the Gln residue in the position corresponding to position 532 of the unprocessed full-length Herpes simplex virus 1 (HSV-1) envelope glycoprotein-B set forth in SEQ ID NO: 1.

10. The modified herpesvirus glycoprotein-B of claim 9, wherein the protein comprises or consists of
(a) the amino acid sequence of SEQ ID NO:7, 8, 19 or 20;
(b) an amino acid sequence which has at least 90% identity to the amino acid sequence of SEQ ID NO:7, 8, 19 or 20;
(c) an immunologically active fragment of any of (a) or (b).

11. A nucleotide sequence encoding the modified herpesvirus envelope glycoprotein-B of claim 1.

12. A plasmid comprising the nucleotide sequence of claim 11.

13. A herpesvirus or recombinant herpesvirus vector comprising the modified herpesvirus envelope glycoprotein-B of claim 1.

14. The herpesvirus or recombinant herpesvirus vector of claim 13, wherein said herpesvirus or recombinant herpesvirus vector is or is derived from HSV-1 or HSV-2.

15. A method of treating a herpesvirus infection comprising administering to a subject in need thereof: (a) the modified herpesvirus glycoprotein-B of claim 1; (b) a nucleotide sequence encoding said modified herpesvirus envelope glycoprotein-B; or (c) a herpesvirus or a recombinant herpesvirus vector comprising said modified herpesvirus envelope glycoprotein-B or said nucleotide sequence.

16. A method of vaccinating a subject against herpesvirus infection comprising administering to a subject in need thereof: (a) the modified herpesvirus glycoprotein-B of claim 1; (b) a nucleotide sequence encoding said modified herpesvirus envelope glycoprotein-B; or (c) a herpesvirus or a recombinant herpesvirus vector comprising said modified herpesvirus envelope glycoprotein-B or said nucleotide sequence.

17. A cell comprising: (a) the modified herpesvirus glycoprotein-B of claim 1; (b) a nucleotide sequence encoding said modified herpesvirus envelope glycoprotein-B; or (c) a herpesvirus or a recombinant herpesvirus vector comprising said modified herpesvirus envelope glycoprotein-B or said nucleotide sequence.

18. A pharmaceutical composition or vaccine comprising: (a) the modified herpesvirus glycoprotein-B of claim 1; (b) a nucleotide sequence encoding said modified herpesvirus envelope glycoprotein-B; or (c) a herpesvirus or a recombinant herpesvirus vector comprising said modified herpesvirus envelope glycoprotein-B or said nucleotide sequence.

19. A method of preparing a vaccine comprising introducing (a) the modified herpesvirus glycoprotein-B of claim 1; (b) a nucleotide sequence encoding said modified herpesvirus envelope glycoprotein-B; or (c) a herpesvirus or a recombinant herpesvirus vector comprising said modified herpesvirus envelope glycoprotein-B or said nucleotide sequence, into a pharmaceutically acceptable carrier.

20. A method of screening for an antiviral compound comprising: incubating a candidate compound with (a) the modified herpesvirus glycoprotein-B of claim 1; (b) a nucleotide sequence encoding said modified herpesvirus envelope glycoprotein-B; (c) a herpesvirus or a recombinant herpesvirus vector comprising said modified herpesvirus envelope glycoprotein-B or said nucleotide sequence; or (d) or a cell comprising any of the foregoing (a)-(c) and detecting interaction of said candidate compound therewith, such as its ability to block viral binding.
